(19) United States
(12) Patent Application Publication
(10) Pub. No.: US 2022/0335092 A1
(43) Pub. Date: Oct. 20, 2022

(54) Literature Information Service Method and Program

(71) Applicants: SHIMADZU CORPORATION, Kyoto-shi (JP); National Institute of Technology and Evaluation, Tokyo (JP); RIKEN, Wako-shi (JP)

(72) Inventors: Yohei YAMADA, Kyoto-shi (JP); Hiroko KAWASAKI, Kisarazu-shi (JP); Akira HOSOYAMA, Tokyo (JP); Seiha MIYAZAWA, Tokyo (JP); Tomokazu SHIRAI, Wako-shi (JP)

(21) Appl. No.: 17/617,182

(22) PCT Filed: Jun. 4, 2020
(86) PCT No.: PCT/JP2020/022206

§ 371 Date: Mar. 14, 2022

(30) Foreign Application Priority Data
Jun. 10, 2019 (JP) 2019-108170

(51) Int. Cl. G06F 16/93; G06F 40/295; G06F 16/903

(52) U.S. Cl. CPC G06F 16/93; G06F 40/295; G06F 16/90335

(57) ABSTRACT

Provided is a literature information service method using a single computer or a plurality of computers connected to each other via a network. The literature information service method includes: transmitting a first character string to a plurality of first servers connected respectively to a plurality of databases each including information of enzymes, and receiving a plurality of pieces of data obtained by searching the plurality of databases with the first character string; extracting a plurality of second character strings indicating information of enzymes from the plurality of pieces of data; generating a search expression using at least one of the plurality of extracted second character strings; and searching a literature database using the search expression to acquire search result data.

### TECHNICAL FIELD

The present invention relates to a literature information service method and a program.

### BACKGROUND ART

When searching a literature database for a patent literature or a non-patent literature such as a research paper, the search is performed by using a search expression including words or phrases. However, since different terms and expressions in the same meaning may be used among literatures, a related literature that does not include those words and phrases included in the search expression may not be searched out, resulting in a search omission. PTL 1 discloses a method of collecting classification codes of patent information included in a literature group obtained as a result of a first search process, and performing a second search to search for a literature including the classification code based on the collected classification codes.

### CITATION LIST

### Patent Literature

PTL 1: Japanese Patent Laying-Open No. 2013-41385

### SUMMARY OF INVENTION

### Technical Problem

Since an enzyme or a gene or the like corresponding to the enzyme is often called by a plurality of different names, a search omission is likely to occur in searching for literatures related to enzymes.

### Solution to Problem

A first aspect of the present invention relates to a literature information service method using a single computer or a plurality of computers connected to each other via a network. The literature information service method includes: acquiring a first character string based on a first input from a user; transmitting the first character string to a plurality of first servers connected respectively to a plurality of databases each including information of enzymes, and receiving a plurality of pieces of data obtained by searching the plurality of databases with the first character string; extracting a plurality of second character strings indicating information of enzymes from the plurality of pieces of data; generating a search expression using at least one of the plurality of extracted second character strings; searching a literature database using the search expression to acquire search result data; and outputting information based on the search result data.

A second aspect of the present invention relates to a program which causes a processor to perform a procedure including: a first character string acquisition process of acquiring a first character string based on an input from a user; a data communication process of transmitting the first character string to a plurality of first servers connected respectively to a plurality of databases each including information of enzymes, and receiving a plurality of pieces of data obtained by searching the plurality of databases with the first character string; a second character string extraction process of extracting a plurality of second character strings indicating information of enzymes from the plurality of pieces of data; a search expression generation process of generating a search expression using at least one of the plurality of extracted second character strings; and a search result data acquisition process of searching a literature database using the search expression to acquire search result data.

### Advantageous Effects of Invention

According the present invention, a search omission in searching for literatures related to enzymes is reduced.

### BRIEF DESCRIPTION OF DRAWINGS

FIG. 1 is a schematic diagram illustrating the configuration of a literature information service system according to an embodiment;

FIG. 2(A) is a schematic diagram illustrating the configuration of a terminal device according to an embodiment;

FIG. 2(B) is a schematic diagram illustrating the configuration of a literature information service server;

FIG. 3 is a schematic diagram illustrating an extracted character string display screen;

FIG. 4 is a schematic diagram illustrating a literature information display screen;

FIG. 5 is a flowchart illustrating a literature information service method according to an embodiment;

FIG. 6(A) is a flowchart illustrating a literature information service method according to an embodiment;

FIG. 6(B) is a flowchart illustrating a literature information service method according to an embodiment;

FIG. 7 is a schematic diagram illustrating the configuration of a literature information service system according to a modification; and

FIG. 8 is a schematic diagram illustrating the distribution of a program.

### DESCRIPTION OF EMBODIMENTS

Hereinafter, embodiments of the present invention will be described with reference to the drawings.

### First Embodiment

The first embodiment describes a literature information service method in which a search expression is generated based on a plurality of pieces of data obtained by searching a plurality of databases including information of enzymes, and the search expression is used to search for a literature from a literature database. In the following embodiments, the “database” is appropriately abbreviated as “DB”.

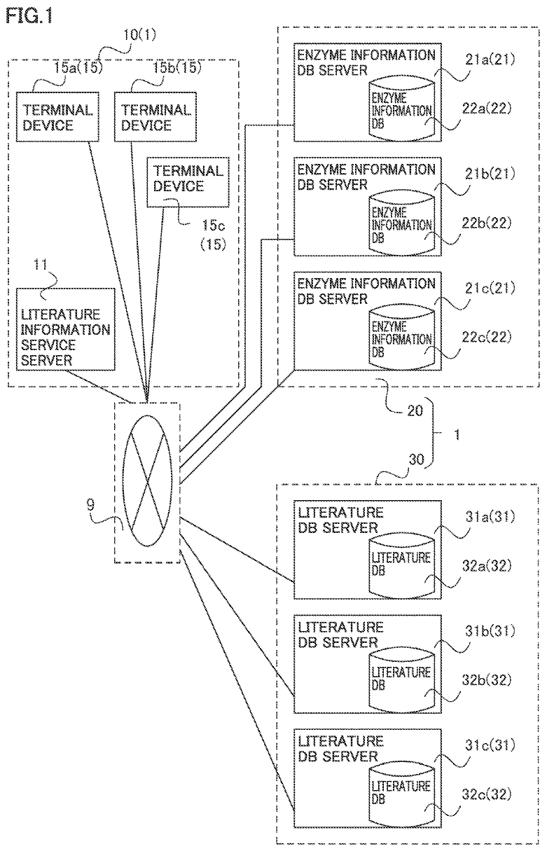

FIG. 1 is a schematic diagram illustrating the configuration of a literature information service system 1 according to the present embodiment. The literature information service system 1 includes a literature information service-side system 10, an enzyme information database-side system (enzyme information DB-side system) 20, and a literature database-side system (literature DB-side system) 30. The literature information service-side system 10 and the enzyme information DB-side system 20 are connected to each other via a network 9, and the literature information service-side system 10 and the literature DB-side system 30 are connected to each other via the network 9.

The network 9 is not particularly limited as long as it may communicate information including at least a character string. The communication of the network 9 is performed according to a communication protocol used in the Internet, such as HTTP (Hypertext Transfer Protocol).

The literature information service-side system 10 includes a literature information service server 11 which is a computer, and a terminal device 15 which is a computer. Although three terminal devices 15a, 15b and 15c are illustrated in FIG. 1, the number of the terminal device 15 is not particularly limited.

The literature information service server 11 and the terminal device 15 are connected to each other via the network 9. Thus, the literature information service server 11 and the terminal device 15 may be arranged at physically separated positions.

The literature information service server 11 and at least one of the terminal devices 15 may be connected to each other via a local network such as a LAN (local area network). The literature information service-side system 10 may be constituted by a single computer.

The literature information service server 11 acquires a character string inputted by a user of the literature information service system 1 (hereinafter, simply referred to as the user) via the terminal device 15. The inputted character string is called an input character string. The literature information service server 11 communicates with the enzyme information DB server 21 and the literature DB server 31, processes data obtained via the communication, and outputs information of a literature searched out from the literature DB 32 to the terminal device 15.

The terminal device 15 functions as an interface configured to receive inputs from a user and transmit outputs to the user. The literature information service server 11 and the terminal device 15 will be described in detail hereinafter.

The enzyme information DB-side system 20 includes an enzyme information database server (enzyme information DB server) 21. The enzyme information DB server 21 includes an enzyme information database (enzyme information DB) 22, and is connected to the enzyme information DB 22 in a searchable manner. Although three enzyme information DB servers 21a, 21b and 21c are illustrated in FIG. 1, the number of enzyme information DB servers 21 is not particularly limited. Although the enzyme information DBs 22a, 22b and 22c are arranged corresponding to the enzyme information DB servers 21a, 21b and 21c, respectively, the number of the enzyme information DBs 22 arranged corresponding to each enzyme information DB server 21 is not particularly limited as long as the number is one or more. The enzyme information DB-side system 20 preferably includes a plurality of enzyme information DBs 22.

The enzyme information DB server 21 receives, from the literature information service server 11, an input character string inputted by the user. The enzyme information DB server 21 searches the enzyme information DB 22 with the input character string, and extracts data including the input character string. The enzyme information DB server 21 transmits the extracted data to the literature information service server 11 as enzyme information search result data.

The communication between the enzyme information DB server 21 and the literature information service server 11 may be performed via another server. The literature information service server 11 and at least one enzyme information DB server 21 may be connected to each other via a local network such as a LAN. In addition, the literature information service server 11 may include a system configured to search at least one enzyme information DB server 21 or the enzyme information DB 22, and the literature information service system 1 may obtain the enzyme information search result data via the system.

The enzyme information DB 22 is a DB including information of enzymes. The information of enzymes includes an enzyme name, an enzyme classification, a gene name corresponding to an enzyme, or a metabolic pathway involving an enzyme (hereinafter, a metabolic pathway involving an enzyme will be simply referred to as the metabolic pathway). The enzyme name, the gene name corresponding to an enzyme, and the metabolic pathway involving an enzyme may have an alternative name used by some skilled persons (hereinafter simply referred to as an alternative name) in addition to a name recommended by a specific organization or the like (hereinafter simply referred to as a recommended name). An example of such an organization includes a collaborative committee consisting of the enzyme committee of the International Union of Biochemistry and Molecular Biology (IUBMB) and the biochemical nomenclature committee of the International Union of Pure and Applied Chemistry (IUPAC). The enzyme classification is preferably classified on the basis of a reaction specificity or a substrate specificity of an enzyme-catalyzed reaction. An example of the enzyme classification is an enzyme commission number (EC number) defined by the collaborative committee. Each EC number specifies an enzyme-catalyzed reaction, and includes four sets of numbers. The enzyme information DB 22 is not particularly limited as long as it includes information of enzymes.

The enzyme information DB 22 may not be an enzyme-specific database as long as it includes information of enzymes. The enzyme information DB 22, for example, may be a database for proteins and/or nucleic acids. The enzyme information DB 22 may be a database integrated with a plurality of databases.

The enzyme information DB 22 includes, for example, molecular information corresponding to a plurality of molecules, respectively. In the molecular information, the information of a molecule is associated with the molecule, and may be referred to by the molecule. The molecular information includes sequence information, structure information, function information or the like of a molecule. The sequence information of a molecule includes an amino acid sequence of a peptide such as a protein, a base sequence of DNA or RNA, and the like. The structure information of a molecule includes a steric arrangement of atoms in the molecule such as a higher order structure of a protein. The molecular information involving the molecular function includes information of a chemical reaction or a metabolic pathway involving the molecule, an interaction with other molecules, and the like.

Hereinafter, the enzyme information DB 22 will be described as a database which stores molecular information corresponding to a plurality of molecules, respectively. Thus, when an input character string is included in an item of molecular information of a molecule, the enzyme information DB server 21 extracts the molecular information. The enzyme information DB server 21 transmits data including the one or more extracted molecular information corresponding to molecules to the literature information service server 11 as enzyme information search result data.

Specific examples of the enzyme information DB 22 include searchable databases such as BRENDA (Braunschweig Enzyme Database), UniProt (Universal Protein Resource), KEGG (Kyoto Encyclopedia of Genes and Genomes), ExPASy-ENZYME (Expert Protein Analysis System-Enzyme nomenclature database), IUBMB Enzyme Nomenclature (International Union of Biochemistry and Molecular Biology) and ExplorEnz.

The literature DB-side system 30 includes one or more literature database servers (literature DB servers) 31. Each literature DB server 31 includes a literature database (literature DB) 32, and is connected to the literature DB 32 in a searchable manner. Although three literature DB servers 31a, 31b, and 31c are illustrated in FIG. 1, the number of the literature DB servers 31 is not particularly limited. Although the literature DBs 32a, 32b and 32c are arranged corresponding to the literature DB servers 31a, 31b and 31c, respectively, the number of the literature DBs 32 arranged corresponding to each literature DB server 31 is not particularly limited as long as it is one or more.

The literature DB server 31 receives, from the literature information service server 11, a search expression generated by a search expression generating unit 126 which will be described later. This search expression is called a literature DB search expression. The literature DB server 31 searches the literature DB 32 with the literature DB search expression, and extracts literatures that meet the conditions of the search expression. The literature DB server 31 transmits data including information indicating the extracted literatures such as data of bibliographic information and the like to the literature information service server 11 as literature search result data.

The communication between the literature DB server 31 and the literature information service server 11 may be performed via another server. The literature information service server 11 and at least one literature DB server 31 may be connected to each other via a local network such as a LAN. In addition, the literature information service server 11 may include a system configured to search at least one enzyme information DB server 21 or the enzyme information DB 22, and the literature information service system 1 may obtain the enzyme information search result data via the system.

The literature DB 32 is not particularly limited as long as it is a database including either the patent literatures or the non-patent literatures such as research papers. As a specific example of the literature DB 32, PubMed may be given.

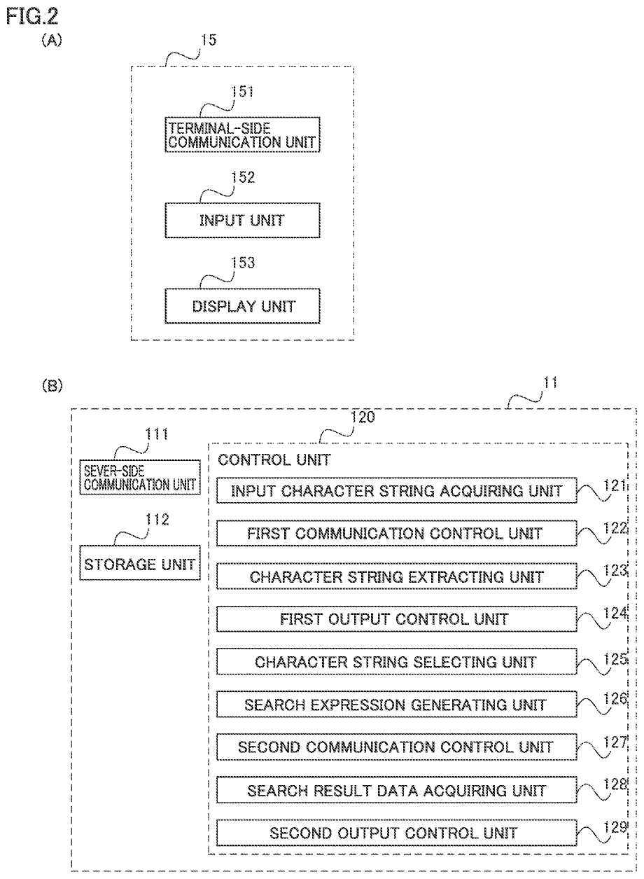

FIG. 2(A) is a schematic diagram illustrating the configuration of the terminal device 15. The terminal device 15 includes a terminal-side communication unit 151, an input unit 152, and a display unit 153. The terminal device 15 is not particularly limited as long as it includes the components illustrated in FIG. 2(A), and it may be constituted by any device which performs inputting/outputting and communication, and may include for example a mobile terminal such as a smartphone or an information processor such as a computer.

The terminal-side communication unit 151 includes a communication device capable of communicating via a wireless connection or a wired connection in accordance with any communication protocol such as a protocol used in the Internet. The terminal-side communication unit 151 communicates with a server-side communication unit 111 in the literature information service server 11 so as to exchange required data.

The input unit 152 includes an input device such as a mouse, a keyboard, various buttons, or a touch panel. The input unit 152 detects an input from a user.

The display unit 153 includes a display device such as a liquid crystal monitor, and displays an input screen and information of search results obtained from the enzyme information DB 22 and the literature DB 32.

FIG. 2(B) is a schematic diagram illustrating the configuration of the literature information service server 11. The literature information service server 11 includes a server-side communication unit 111, a storage unit 112, and a control unit 120. The control unit 120 includes an input character string acquiring unit 121, a first communication control unit 122, a character string extracting unit 123, a first output control unit 124, a character string selecting unit 125, a search expression generating unit 126, a second communication control unit 127, a search result data acquiring unit 128, and a second output control unit 129.

The server-side communication unit 111 includes a communication device capable of communicating via a wireless connection or a wired connection in accordance with any communication protocol such as a protocol used in the Internet. The server-side communication unit 111 communicates with the terminal device 15, the enzyme information DB server 21, and the literature DB server 31 so as to exchange required data.

The storage unit 112 includes a nonvolatile storage medium. The storage unit 112 stores data required for the processing of the control unit 120, data obtained by the processing of the control unit 120, programs required by the control unit 120 to perform the processing, and the like.

The control unit 120 includes a processor such as a CPU, and functions to control the literature information service server 11. The control unit 50 performs various processing by executing programs stored in the storage unit 112 or the like.

The input character string acquiring unit 121 of the control unit 120 acquires an input character string inputted by the user. The input character string is preferably a character string corresponding to the enzyme name or the enzyme classification. In the case of the enzyme classification, the classification is more preferably classified on the basis of the reaction specificity or substrate specificity of an enzyme reaction catalyzed by an enzyme having an EC number described above.

The inputting method of an input character string by the user is not particularly limited. For example, the user may use a keyboard to type an input character string into a text box of an input screen displayed on the display unit 153 of the terminal device 15 and use a mouse to click a send button or the like. Alternatively, a text file including input character strings may be transmitted from the terminal device 15 to the literature information service server 11 and stored in the literature information service server 11, and the input character string acquiring unit 121 may read an input character string from the text file based on the user input.

The input character string acquiring unit 121 stores the input character string obtained from the user input in the storage unit 112 or the control unit 120, and sets the input character string in such a state that the input character string may be referred to by a reference instruction from the control unit 120 (hereinafter referred to as “stored in the storage unit 112 or the like in a referenceable manner”).

The first communication control unit 122 controls the server-side communication unit 111 to communicate with the enzyme information DB server 21. The first communication control unit 122 transmits an input character string to the enzyme information DB server 21. The first communication control unit 122 receives, from the enzyme information DB server 21, the enzyme information search result data obtained as a search result based on the transmitted input character string.

The character string extracting unit 123 extracts a character string from the enzyme information search result data. The character string extracted by the character string extracting unit 123 is called an extracted character string. The extracted character string is a character string corresponding to the information of enzymes described above. The character string extracting unit 123 refers to the items indicating the enzyme name, the enzyme classification, the gene name corresponding to the enzyme, or the like in the enzyme information search result data, and extracts a character string corresponding to those items. The character string extracting unit 123 may extract a character string corresponding to a prefix, a suffix, or the like. For example, since the enzyme number has a feature that “EC” is followed by a series of numbers, the extracted character string may be extracted based on this feature.

Note that the character string extracting unit 123 may refer to the item indicating a metabolic pathway of an enzyme, and may extract a character string corresponding to the item indicating the metabolic pathway of the enzyme.

The character string extracting unit 123 stores the extracted character strings in the storage unit 112 or the like in a referenceable manner. When the extracted character strings are associated with each other, the character string extracting unit 123 stores information of association (hereinafter referred to as the association information) in the storage unit 112 in a referenceable manner. The character string extracting unit 123 stores, in the storage unit 112 or the like in a referenceable manner, information indicating a database serving as an information source of the data from which the extracted character strings are extracted.

The character string extracting unit 123 sorts the extracted character strings as necessary based on the association information, and generates data for constructing a list of extracted character strings (hereinafter referred to as the list data). In the list data, the extracted character strings such as the enzyme name, the gene name, and the like are associated with the extracted character strings such as the classification of each enzyme number (EC number) and the like based on the association information. The enzyme name and the gene name may include a variety of different names that refer to the same enzyme or gene, such as a synonym or an abbreviation. When creating the list data, the character string extracting unit 123 may appropriately execute processes such as distinguishing a recommended name and an alternative name which will be described later based on data stored in advance, deleting the other extracted character strings and leaving one extracted character string if there are a plurality of the same extracted character strings, or sorting the extracted character strings in a predefined order. In the list data, the enzyme name and the gene name are associated with information indicating a database serving as an information source from which the enzyme name and the gene name are extracted. The character string extracting unit 123 stores the list data in the storage unit 112 or the like in a referenceable manner.

When the metabolic pathway of an enzyme is extracted as the extracted character string, the character string extracting unit 123 may, based on the association information, associate the extracted character string of the metabolic pathway with the enzyme number or the information indicating the database serving as the information source. Thus, when the metabolic pathway is extracted as an extracted character string, the same process may be performed as that to be performed when the enzyme name or the like is extracted as the extracted character string, which will be described hereinafter.

The first output control unit 124 control the output of an extracted character string. The first output control unit 124 generates data for displaying a list (hereinafter referred to as the list display data) from the list data. The format of the list display data is not particularly limited as long as an image of the list may be displayed on the terminal device 15 and a user may input for selection of a character string by the character string selecting unit 125 which will be described later. When the network 9 supports the HTTP communication protocol, the list display data may be implemented as an HTML file, an XML file or the like, and an image indicating the list may be displayed on the display unit 153 of the terminal device 15 via a Web browser.

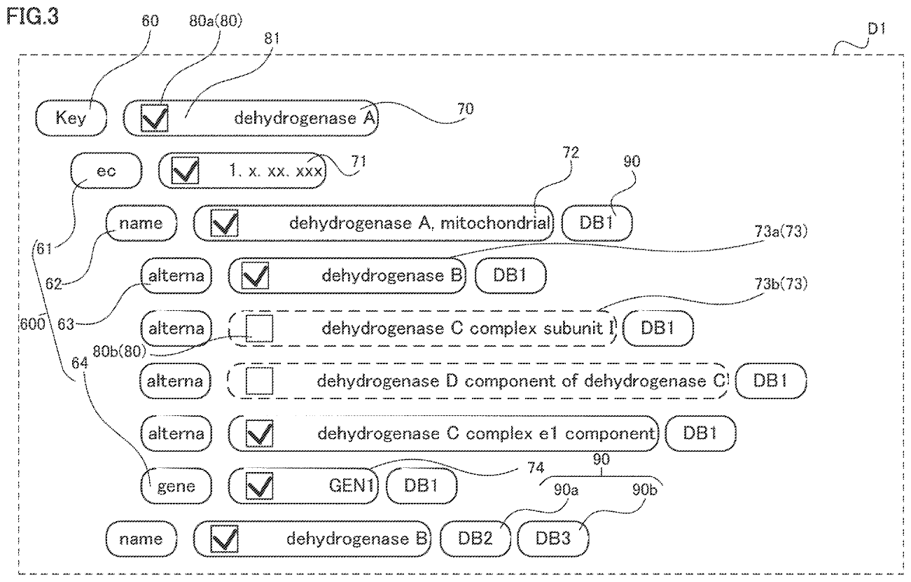

FIG. 3 is a schematic diagram illustrating an example of an extracted character string list display screen displayed on the terminal device 15 under the control of the first output control unit 124. FIG. 3 illustrates an example in which the input character string is “dehydrogenase A”.

The extracted character string list display screen D1 includes an input character string item 60, an enzyme information item 600, an input character string display item 70, a classification display item 71, a name display item 72, an alternative name display item 73, a gene name display item 74, a switching item 80, and a DB display item 90. The enzyme information item 600 includes a classification item 61, a name item 62, an alternative name item 63, and a gene name item 64.

The input character string item 60 indicates that the information displayed in association with the item is an input character string by using the term “Key”. The enzyme information item 600 indicates that the information displayed in association with the item is information of an enzyme. The classification item 61 indicates that the information displayed in association with the item is an enzyme classification (EC number in this case) by using the term “ec”. The name item 62 indicates that the information displayed in association with the item is a recommended name of an enzyme by using the term “name”. In the present embodiment, the recommended name may be, for example, a name recommended by a specific organization such as the IUBMBAUPAC Committee. The alternative name item 63 indicates that the information displayed in association with the item is an alternative name of an enzyme other than the recommended name by using the term “alterna” (abbreviation of alternative name). The gene name item 64 indicates that the information displayed in association with the item is a gene name corresponding to the enzyme by using the term “gene”.

The name item 62 may indicate any representative name such as a name initially displayed in the search result of each enzyme information DB 22 instead of the recommended name. Such name may be a single name such as a recommended name by the IUBMBAUPAC Committee, or may be a plurality of representative names.

The input character string display item 70 is arranged in the same row in association with the input character string item 60 to display an input character string. In the example of FIG. 3, the input character string is displayed as the enzyme name “dehydrogenase A”. The classification display item 71 is arranged in the same row in association with the classification item 61 to display an enzyme classification as an extracted character string. In the example of FIG. 3, the enzyme classification is displayed as an EC number of 1.x.xx.xxx (x, xx and xxx are digits) extracted in association with the input character string.

The name display item 72 is arranged in the same row in association with the name item 62 to display the recommended enzyme name as the extracted character string. In the example of FIG. 3, the recommended enzyme name is displayed as the enzyme name extracted in association with the enzyme number displayed in the classification display item 71. The alternative name display item 73 is arranged in the same row in association with the alternative name item 63 to display an alternative name of the enzyme as the extracted character string. In the example of FIG. 3, the alternative name of the enzyme is displayed as an enzyme name different from the recommended name extracted in association with the EC number displayed in the classification display item 71. The gene name display item 74 is arranged in the same row in association with the gene name item 64 to display the gene name corresponding to the enzyme which is the extracted character string. In the example of FIG. 3, the gene name of the enzyme is displayed as the gene name extracted in association with the EC number displayed in the classification display item 71.

The switching item 80 is an icon arranged in the same row in association with each extracted character string to switch whether or not to use the extracted character string to generate a literature DB search expression which will be described later. In the example of FIG. 3, the switching item 80 is displayed as a check box. The switching item 80 is configured in such a manner that when the check box is checked (ON, see for example the switching item 80a), a literature DB search expression is generated by using the extracted character string, and when the check box is not checked (OFF, see for example the switching item 80b), a literature DB search expression is generated without using the extracted character string. The user may switch the switching item 80 by clicking the check box with a mouse or the like.

The switching item 80 is not particularly limited as long as the user may switch whether or not to use the extracted character string to generate a literature DB search expression.

For example, if an extracted character string in the list of extracted character strings is considered to be less related to the enzyme corresponding to the input character string, the user may use the switching item 80 to exclude the extracted character string from the literature DB search expression so as to avoid extracting unnecessary literatures.

In FIG. 3, when the switching item 80 is ON, the alternative name display item 73a is surrounded by a solid line, and when the switching item 80 is OFF, the alternative name display item 73b is surrounded by a broken line. As described above, the display mode of an extracted character string may be changed depending on whether or not to use the extracted character string to generate a literature DB search expression.

The DB display item 90 is arranged in the same row in association with each extracted character string to display a database as an information source of the extracted character string. In the example of FIG. 3, the names of databases serving as the information sources are indicated by “DB1”, “DB2”, “DB3”, and the like. When an extracted character string is extracted+ from a plurality of databases, a plurality of DB display items 90a and 90b may be displayed in association with the extracted character string.

The metabolic pathway may be displayed in the same manner as the extracted character string, and may be displayed in association with the switching item 80 and the DB display item 90.

In the extracted character string list display screen D1, information of each extracted character string is associated to each other by being displayed in the same row. A plurality of extracted character strings associated with a certain enzyme number are associated with the extracted character string by being displayed collectively below the classification display item 71 displaying the enzyme number. Thus, it is preferable to sort and display each extracted character string based on the enzyme classification such as the enzyme number. The sorting method is not particularly limited. The shape and the position of each item are not particularly limited as long as the user may know the association of each item on the extracted character string list display screen D1.

The character string selecting unit 125, based on an input from the user, selects at least one character string from the extracted character strings to generate a literature DB search expression. The character string selected by the character string selecting unit 125 is called a selected character string. The user operates the input unit 152 of the terminal device 15 by clicking a send button (not shown) on the extracted character string list display screen D1 to cause the terminal-side communication unit 151 to transmit information related to the switching of the switching item 80 for each extracted character string (hereinafter referred to as the switching information) to the literature information service server 11.

When the extracted character string includes a metabolic pathway, the metabolic pathway may be used as the selected character string.

The character string selecting unit 125 selects a selected character string based on the switching information received by the server-side communication unit 111. The character string selecting unit 125 stores the selected character string in the storage unit 112 or the like in a referenceable manner.

The search expression generating unit 126 generates, from the selected character string, a literature DB search expression for searching the literature DB 32. The method of generating a literature DB search expression is not particularly limited as long as the search expression may be generated from the selected character string. However, from the viewpoint of preventing search omission, it is preferable to use a logical sum (OR) of each selected character string within each category of the enzyme name, the enzyme classification and the gene name.

It should be noted that when the metabolic pathway is included in the selected character string, the search expression generating unit 126 may also use a logical sum of the selected character string within the category of the metabolic pathway in the same manner. Similarly, the following process of generating a literature DB search expression is applied to the metabolic pathway.

For example, suppose that the enzyme names A1 and A2, the enzyme classifications B1, B2 and B3, the gene names C1, C2, C3 and C4, and the metabolic pathways D1 and D2 are selected as the selected character strings. In this case, as an example, the search expression generating unit 126 may generate a literature DB search expression “(A1 OR A2) AND (B1 OR B2 OR B3) AND (C1 OR C2 OR C3 OR C4) AND (D1 OR D2)”. The search range may be made wider by using OR instead of AND between each category of the selected character strings.

The search expression generating unit 126 may acquire a character string inputted by the user via the terminal device 15 (hereinafter referred to as the additional character string), and generate a search expression based on the additional character string. For example, the search expression generating unit 126 may combine the additional character string with the literature DB search expression by an arbitrary logical operation formula including AND or OR. The additional character string may be a plurality of character strings.

At the time of generating a literature DB search expression, after a literature DB search expression is created, the search expression may be modified according to an instruction received from the user so as to search a narrower or wider range, or a search expression for searching various ranges may be created and stored in advance.

The second communication control unit 127 controls the server-side communication unit 111 to communicate with the literature DB server 31. The second communication control unit 127 transmits the literature DB search expression to the literature DB server 31. The literature DB search expression may be complied in accordance with the specifications of each literature DB server 31 without changing the search result. The second communication control unit 127 receives the literature search result data obtained as a search result using the transmitted literature DB search expression.

The search result data acquiring unit 128 stores the literature search result data in the storage unit 112 or the like in a referenceable manner.

The second output control unit 129 controls the output of the literature information obtained as a search result using the literature DB search expression. The second output control unit 129 generates data for displaying a searched literature (hereinafter referred to as the literature display data) on the basis of the literature search result data. The format of the literature display data is not particularly limited as long as the bibliographic information and the like of a searched literature may be displayed on the terminal device 15. When the network 9 supports the HTTP communication protocol, the literature display data may be implemented as an HTML file, an XML file, or the like, and an image indicating the bibliographic information and the like of the searched literature may be displayed on the display unit 153 of the terminal device 15 via a Web browser.

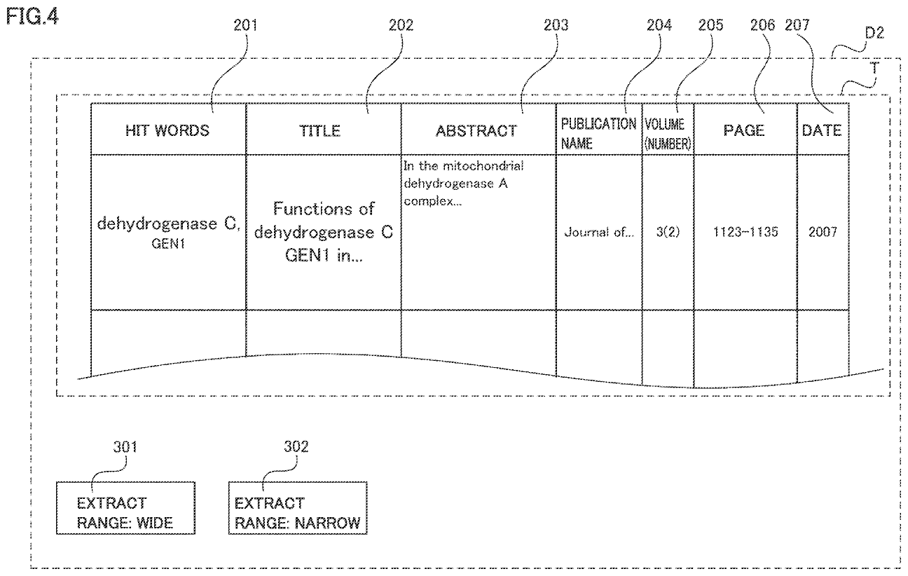

FIG. 4 is a schematic diagram illustrating an example literature information display screen displayed on the terminal device 15 under the control of the second output control unit 129. The literature information display screen D2 includes a table T and extract range switching icons 301 and 302.

If the literature DB search expression is created based on the selected character string so as to search the literature DB, the extract range may not be switched. For example, the user may specify an extract range, a literature DB search expression may be created based on the specified extract range to search for literatures, and the searched literatures may be displayed. At the time of switching the extract range, the user may specify the extract range again, and the flow mentioned above may be repeated. Alternatively, instead of displaying the extract range switching icons 301 and 302, the function of the extract range switching icons 301 and 302 may be implemented by another method such as switching by an input from a keyboard or the like.

The table T of the literature information display screen D2 includes a selected character string item 201, a title item 202, an abstract item 203, a publication name item 204, a volume & number item 205, a page item 206, and a publication date item 207.

The information included in the literature information display screen D2 is not particularly limited as long as it is possible to specify the searched literature. In the example of FIG. 4, the abstract of a non-patent literature such as a research paper is displayed, but it is possible to display a patent literature. Furthermore, the arrangement of the publication name item 204, the volume & number item 205, and the page item 206 is not particularly limited as long as it is possible to specify the searched literature, and for example, they may be displayed in the same column as the title.

The selected character string item 201 indicates that which one of the selected character strings in the literature DB search expression is used to extract the searched literature. In the example of FIG. 4, two of the selected character strings “dehydrogenase C” and “GEN1” are used to extract the searched literature. In the present embodiment, “extracted in association with the selected character string” means that the selected character string is included in the search range of searching the literature DB 32. The search range may be appropriately defined based on the title, the abstract, the full text, and the like. In this way, the literature information display screen D2 displays the information of the searched literature in association with the information of enzymes corresponding to the selected character string, based on the literature search result data.

The title item 202 indicates the title of the searched literature. The abstract item 203 indicates the abstract of the searched literature. The publication name item 204 indicates the name of a publication including the searched literature. The volume & number item 205 indicates the volume and number of the publication including the searched literature. The page item 206 indicates pages of the searched literature in the publication. The publication date item 207 indicates the date at which the publication including the searched literature is published or released online.

The extract range switching icons 301 and 302 are icons used to switch an extract range for extracting literatures to be displayed on the literature information display screen D2 from the literature search result data based on the literature DB search expression. The extract range switching icon 301 is used to display a literature search result based on a search expression corresponding to an extract range wider than the extract range switching icon 302.

For example, suppose that the enzyme names A1 and A2, the enzyme classifications B1, B2 and B3, the gene names C1, C2, C3 and C4, and the metabolic pathways D1 and D2 are selected as the selected character strings. In this case, for example, when the user clicks the extract range switching icon 301, it is possible to display a literature search result based on the literature DB search expression of “(A1 OR A2) OR (B1 OR B2 OR B3) OR (C1 OR C2 OR C3 OR C4) OR (D1 OR D2)”. When the user clicks the extract range switching icon 302, it is possible to display a literature search result based on the literature DB search expression of “(A1 OR A2) AND (B1 OR B2 OR B3) AND (C1 OR C2 OR C3 OR C4) AND (D1 OR D2)”.

In order to acquire a literature search result based on a plurality of different literature DB search expressions, each search expression may be used as a literature DB search expression to search the literature DB 32 and the obtained search result is communicated via communication. Alternatively, based on the selected character string associated with each literature of the acquired literature search result data, the literature information service server 11 may generate search result data based on a search expression corresponding to a different extract range. In other words, the literature information service server 11 may record the created literature DB search expression and the literature search result (associated with the selected character string), and process the previous data and use the processed data to perform a new literature search.

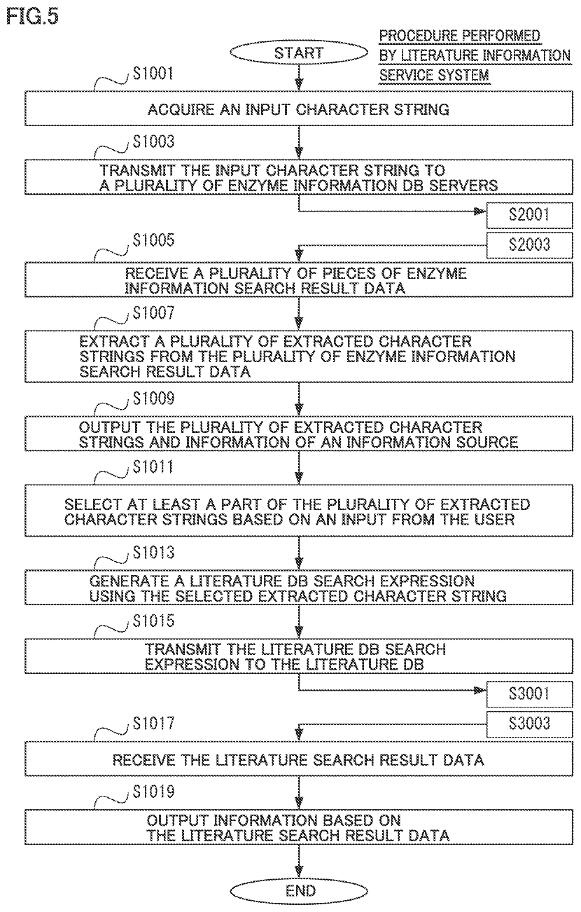

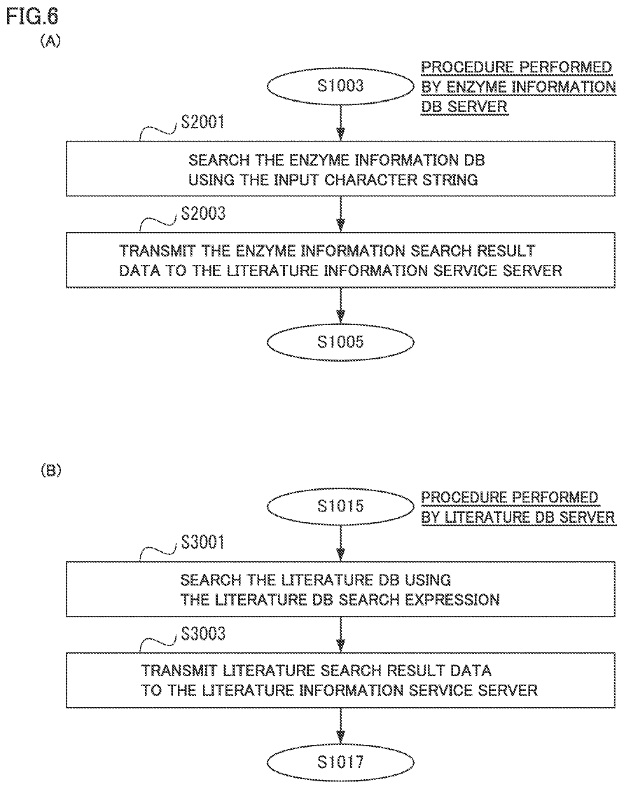

FIGS. 5, 6(A) and 6(B) are flowcharts illustrating a literature information service method according to the present embodiment. FIG. 5 illustrates a procedure performed by the literature information service-side system 10. In step S1001, the input character string acquiring unit 121 acquires an input character string. Upon completion of step S1001, step S1003 is started. In step S1003, the first communication control unit 122 controls the server-side communication unit 111 to transmit the input character string to a plurality of enzyme information DB servers 21. Upon completion of step S1003, step S2001 is started.

FIG. 6(A) illustrates a procedure performed by the enzyme information DB-side system 20. In step S2001, the enzyme information DB server 21 searches the enzyme information DB 22 using the input character string. Upon completion of step S2001, step S2003 is started. In step S2003, the enzyme information DB server 21 transmits the enzyme information search result data to the literature information service server 11. Upon completion of step S2003, step S1005 is started.

In step S1005 (FIG. 5), the first communication control unit 122 controls the server-side communication unit 111 to receive a plurality of enzyme information search result data. Upon completion of step S1005, step S1007 is started. In step S1007, the character string extracting unit 123 extracts a plurality of character strings from the plurality of enzyme information search result data, and creates a list of the extracted character strings. Upon completion of step S1007, step S1009 is started.

In step S1009, the first output control unit 124 outputs data indicating the plurality of extracted character strings and information of an information source DB to the terminal device 15, and the extracted character string list display screen D1 is displayed on the display unit 153. Upon completion of step S1009, step S1011 is started. In step S1011, the character string selecting unit 125 selects at least a part of the plurality of extracted character strings based on an input from the user. Upon completion of step S1011, step S1013 is started.

In step S1013, the search expression generating unit 126 generates a literature DB search expression using the selected extracted character string. Upon completion of step S1013, step S1015 is started. In step S1015, the second communication control unit 127 controls the server-side communication unit 111 to transmit the literature DB search expression to the literature DB 31. Upon completion of step S1015, step S3001 is started.

FIG. 6(B) illustrates a procedure performed by the literature DB-side system 30. In step S3001, the literature DB server 31 searches the literature DB 32 using the literature DB search expression. Upon completion of step S3001, step S3003 is started. In step S3003, the literature DB server 31 transmits the literature search result data to the literature information service server 11. Upon completion of step S3003, step S1017 is started.

In step S1017 (FIG. 5), the second communication control unit 127 controls the server-side communication unit 111 to receive the literature search result data. Upon completion of step S1017, step S1019 is started. In step S1019, the second output control unit 129 outputs information based on the literature search result data, and the information is displayed on the display unit 153. Upon completion of step S1019, the procedure is ended.

The following modifications are within the scope of the present invention and may be combined with the embodiment described above. In the following modifications, the components having the same structure and functions as those in the embodiment described above will be denoted by the same reference numerals, and the description thereof will not be repeated.

(First Modification)

In the embodiment described above, it is described that the enzyme information DB server 21 may search the enzyme information DB 22 in the past or acquire information of the data change history of the enzyme information DB 22. Thus, the literature information service server 11 may acquire the enzyme information search result data obtained by searching the enzyme information DB 22 in the past based on the input character string, or the enzyme information search result data based on the data change history. This makes it possible to cover the contents of the enzyme information DB 22 in the past so as to reduce the search omission of literatures related to enzymes.

In the present modification, when transmitting the input character string to the enzyme information DB server 21, the first communication control unit 122 appropriately transmits information on the condition related to the search range so as to obtain the search result from the enzyme information DB 22 in the past.

(Second Modification)

In the embodiment described above, it is described that the literature information service-side system 10 is constructed by the literature information service server 11 and the terminal device 15. However, the literature information service-side system may be constructed by an information processor or an analysis device including the information processor.

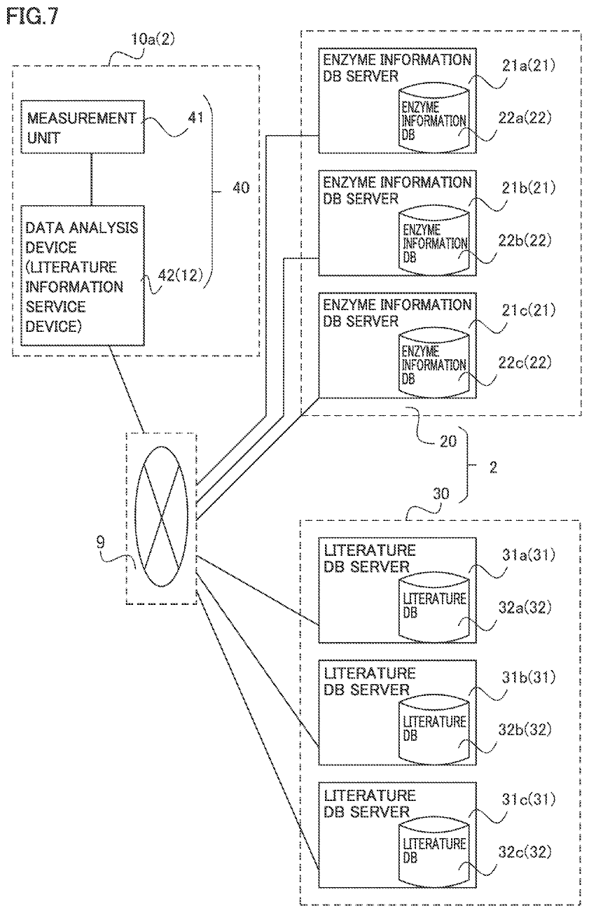

FIG. 7 is a schematic diagram illustrating the configuration of a literature information service system 2 according to the present modification. The literature information service system 2 includes a literature information service-side system 10a, an enzyme information DB-side system 20, and a literature DB-side system 30.

The literature information service-side system 10a includes an analysis device 40, and the analysis device 40 includes a measurement unit 41 and a data analysis device 42. The analysis device 40 is not particularly limited, and it may include a separation & analysis device. The separation & analysis device is not particularly limited, and it may include at least one of a chromatograph and a mass spectrometer.

The measurement unit 41 performs physical or chemical analysis on a sample to obtain measurement data. The data analysis device 42 includes an information processor such as a computer to analyze the measurement data, and constitutes the literature information service device 12 which performs the literature information service method of the present modification.

The data analysis device 42 communicates with the enzyme information DB server 21 and the literature DB server 31 of the server-side communication unit 111, and includes a storage unit 112, an input unit 152, a display unit 153, and a control unit 120.

It is not necessary for the literature information service device 12 to be a part of the analysis device 40, and it may be an information processor such as a computer or a mobile terminal separated from the measurement unit 41.

(Third Modification)

A program for implementing the information processing function of the literature information service server 11 or the literature information service device 12 may be recorded in a computer-readable recording medium, and the program recorded in the recording medium in association with the processing by the control unit 120 and the control of the processing related thereto may be read and executed by a computer system. The “computer system” includes an OS (Operating System) and peripheral devices as hardware. The “computer-readable recording medium” refers to a portable recording medium such as a flexible disk, a magneto-optical disk, an optical disk, or a memory card, or a storage device such as a hard disk built in the computer system. In addition, the “computer-readable recording medium” may include a medium that dynamically retains a program for a short time, such as a communication line that transmits the program via a network such as the Internet or a telephone line, and a medium that retains the program for a fixed time, such as a volatile memory in a computer system serving as a server or a client. The program described above may implement a part of the functions described above, or may implement the functions described above in combination with a program previously recorded in the computer system.

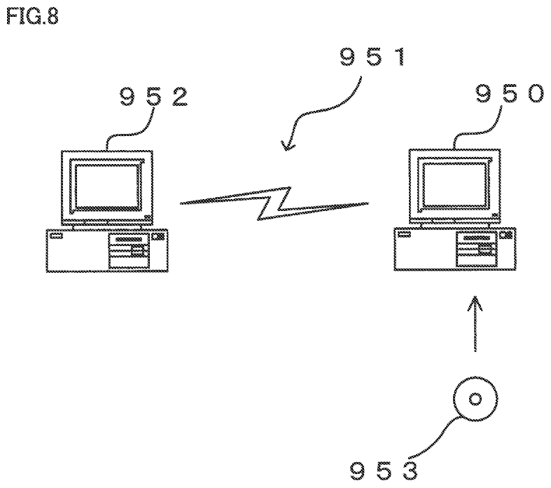

When the present invention is applied to a personal computer (hereinafter referred to as “PC”) or the like, the program related to the above-described control may be distributed via a recording medium such as a CD-ROM or a DVD-ROM or via a data signal through the Internet or the like, which is illustrated in FIG. 8. A PC 950 receives a program via a CD-ROM 953. The PC 950 has a function of connecting to a communication line 951. A computer 952 is a server computer that distributes the program, and stores the program in a recording medium such as a hard disk. The communication line 951 is a communication line such as the Internet or personal computer communication, or a dedicated communication line. The computer 952 reads the program from the hard disk and transmits the program to the PC 950 via the communication line 951. In other words, the program is carried by a carrier wave as a data signal and transmitted via the communication line 951. As described above, the program may be distributed as a computer program product readable by a computer in various forms such as a recording medium and a carrier wave.

(Fourth Modification)

In the embodiment described above, the processes performed by the control unit 120, such as the processes performed by the first communication control unit 122, the character string extracting unit 123, the first output control unit 124, the character string selecting unit 125, the search expression generating unit 126, the second communication control unit 127, and the search result data acquiring unit 128 may be performed by an information processor such as a PC having a processor or a control unit disposed in the terminal device 15 including the information processor. In this case, the program for performing these processes may be provided to the terminal device 15 as in the third modification.

According to the embodiment described above or the modifications, the following effects may be obtained.

(1) In an embodiment according to the first aspect, a literature information service method is a literature information service method using a single computer or a plurality of computers connected to each other via a network. The literature information service method includes: acquiring a first character string based on a first input from a user; transmitting the first character string to a plurality of first servers connected respectively to a plurality of databases each including information of enzymes, and receiving a plurality of pieces of data obtained by searching the plurality of databases with the first character string; extracting a plurality of second character strings indicating information of enzymes from the plurality of pieces of data; generating a search expression using at least one of the plurality of extracted second character strings; searching a literature database using the search expression to acquire search result data; and outputting information based on the search result data. This makes it possible to reduce a search omission in searching for literatures related to enzymes.

(2) In an embodiment according to the second aspect, the literature information service method according to the first aspect further includes, as processes to be performed by a computer, displaying the plurality of extracted second character strings after the extract of the plurality of second character strings, detecting a second input from the user for the plurality of second character strings, and generating the search expression using at least one character string among the plurality of extracted second character strings, the at least one character string being searched out based on the second input. Thus, the character string used in a search expression for searching for a literature may be selected based on an input from the user, which makes it possible to obtain a search result with higher accuracy.

(3) In an embodiment according to the third aspect, the literature information service method according to the first aspect or the second aspects further includes, as processes to be performed by a computer, associating information of the first server or the database serving as an information source with each of the plurality of extracted second character strings. Thus, the character string used in a search expression for searching a literature may be provided to a user together with the information of a database serving as an information source.

(4) In an embodiment of the fourth aspect, in the literature information service method according to any one of the first to third aspects, information of a searched literature is output by a computer in association with information of enzymes based on the search result data. Thus, it is possible to clearly understand that the literature is associated with which enzyme by the terms of the name of an enzyme or a corresponding gene.

(5) In an embodiment of the fifth aspect, in the literature information service method of any one of the first to fourth aspects, the first character string is a character string corresponding to an enzyme name or an enzyme classification. Although the same enzyme or a gene corresponding to the same enzyme is often called by a plurality of different names, a search result covering these names may be obtained by this configuration.

(6) In an embodiment of the sixth aspect, in the literature information service method of any one of the first to fifth aspects, the information of enzymes is at least one of an enzyme name, an enzyme classification, a gene name, and a metabolic pathway. Thus, it is possible to reduce a search omission of literatures related to the enzyme name, the enzyme classification, the gene name and the metabolic pathway.

(7) In an embodiment of the seventh aspect, in the literature information service method according to any one of the first to sixth aspects, the enzyme classification is classified on the basis of a reaction specificity and a substrate specificity. Thus, it is possible to reduce the search omission of related literatures as described above based on the reaction specificity and the substrate specificity of the enzymatic reaction.

(8) In an embodiment of the eighth aspect, the program is a program which causes a processor to perform a procedure including: a first character string acquisition process (corresponding to step S1001 in the flowchart of FIG. 5) of acquiring a first character string based on an input from a user; a data communication process (corresponding to steps S103 and S1005) of transmitting the first character string to a plurality of first servers connected respectively to a plurality of databases each including information of enzymes, and receiving a plurality of pieces of data obtained by searching the plurality of databases with the first character string; a second character string extraction process (corresponding to step S1007) of extracting a plurality of second character strings indicating information of enzymes from the plurality of pieces of data; a search expression generation process (corresponding to step S1013) of generating a search expression using at least one of the plurality of extracted second character strings; and a search result data acquisition process (corresponding to step S1017) of searching a literature database using the search expression to acquire search result data. This makes it possible to reduce search omission in searching literatures related to enzymes.

The present invention is not limited to the embodiments mentioned above. Other embodiments considered within the scope of the technical idea of the present invention are also included within the scope of the present invention.

The disclosure of the following priority application is incorporated herein by reference.
Japanese Patent Laying-Open No. 2019-108170 (filed on Jun. 10, 2019).

### REFERENCE SIGNS LIST

1, 2: literature information service system; 9; network; 10, 10a: literature information service-side system; 11: literature information service server; 12: literature information service device; 15, 15a, 15b, 15c: terminal device; 20: enzyme information DB-side system; 21, 21a, 21b, 21c: enzyme information DB server; 22, 22a, 22b, 22c: enzyme information DB; 30: literature DB-side system; 31a, 31b, 31c: literature DB server; 32, 32a, 32b, 32c: literature DB; 40: analysis device; 42: data analysis device; 60: input character string item; 61: classification item; 62: name item; 63: alternative name item; 64: gene name item; 70: input character string display item; 71: classification display item; 72: name display item; 73: alternative name display item; 74: gene name display item; 80, 80a, 80b: switching item; 90, 90a, 90b: DB display item; 121: input character string acquiring unit; 122: first communication control unit; 123: character string extracting unit; 124: first output control unit; 125: character string selecting unit; 126: search expression generating unit; 127: second communication control unit; 128: search result data acquiring unit; 129: second output control unit; D1: extracted character string list display screen; D2: literature information display screen

1. A literature information service method using a single computer or a plurality of computers connected to each other via a network, the literature information service method comprising:
acquiring a first character string based on a first input from a user;
transmitting the first character string to a plurality of first servers connected respectively to a plurality of databases each including information of enzymes, and receiving a plurality of pieces of data obtained by searching the plurality of databases with the first character string;
extracting a plurality of second character strings indicating information of enzymes from the plurality of pieces of data;
generating a search expression using at least one of the plurality of extracted second character strings;
searching a literature database using the search expression to acquire search result data; and
outputting information based on the search result data.

2. The literature information service method according to claim 1, further comprising:
displaying the plurality of extracted second character strings after the extract of the plurality of second character strings;
detecting a second input from the user for the plurality of second character strings; and
generating the search expression using at least one character string among the plurality of extracted second character strings, the at least one character string being searched out based on the second input.

3. The literature information service method according to claim 1, further comprising:
associating information of the first server or the database which serves as an information source with each of the plurality of extracted second character strings.

4. The literature information service method according to claim 1, wherein
information of a searched literature is output in association with the information of enzymes based on the search result data.

5. The literature information service method according to claim 1, wherein
the first character string is a character string corresponding to an enzyme name or an enzyme classification.

6. The literature information service method according to claim 1, wherein
the information of enzymes is at least one of an enzyme name, an enzyme classification, a gene name, and a metabolic pathway involving the enzyme.

7. The literature information service method according to claim 1, wherein
the enzyme classification is classified on the basis of a reaction specificity and a substrate specificity.

8. A program which causes a processor to perform a procedure including:
a first character string acquisition process of acquiring a first character string based on an input from a user;
a data communication process of transmitting the first character string to a plurality of first servers connected respectively to a plurality of databases each including information of enzymes, and receiving a plurality of pieces of data obtained by searching the plurality of databases with the first character string;
a second character string extraction process of extracting a plurality of second character strings indicating information of enzymes from the plurality of pieces of data;
a search expression generation process of generating a search expression using at least one of the plurality of extracted second character strings; and
a search result data acquisition process of searching a literature database using the search expression to acquire search result data.
